(19) United States
(12) Patent Application Publication
(10) Pub. No.: US 2013/0317134 A1
(43) Pub. Date: Nov. 28, 2013

(54) TISSUE ADHESIVE BASED ON TRIFUNCTIONAL ASPARTATES

(71) Applicants: Heike Heckroth, Odenthal (DE); Christoph Eggert, Koln (DE)

(72) Inventors: Heike Heckroth, Odenthal (DE); Christoph Eggert, Koln (DE)

(73) Assignee: Bayer Intellectual Property GmbH Creative Campus Monheim, Monheim (DE)

(21) Appl. No.: 13/984,170

(22) PCT Filed: Feb. 6, 2012
(86) PCT No.: PCT/EP2012/051934

§ 371 Date: Aug. 7, 2013

(30) Foreign Application Priority Data
Feb. 9, 2011 (EP) 11153807.0

(51) Int. Cl. A61L 24/04

(52) U.S. Cl. CPC A61L 24/046
USPC 523/118; 560/169; 137/602

(57) ABSTRACT

The present invention relates to a compound of formula (I) wherein R₁, R₂ each independently of the other are identical or different organic radicals which do not contain Zerewitinoff-active hydrogen, and R₄, R₅, R₆ each independently of the others are saturated, linear or branched organic radicals which do not contain Zerewitinoff-active hydrogen and which are also optionally substituted in the chain by heteroatoms, for use in a polyurea system which is provided in particular for sealing, bonding, gluing or covering cell tissue. The invention further provides a polyurea system comprising the compound according to the invention, and a metering system for the polyurea system according to the invention.

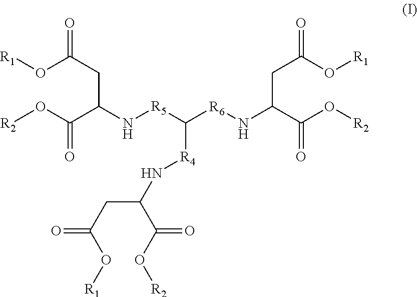

The present invention relates to an amino-functional compound for use in a polyurea system which is provided in particular for the sealing, bonding, gluing or covering of cell tissue. The invention further provides a polyurea system comprising the compound according to the invention, and a metering system for the polyurea system according to the invention.

Various materials which are used as tissue adhesives are available commercially. They include the cyanoacrylates Dermabond® (octyl 2-cyanoacrylate) and Histoacryl Blue® (butyl cyanoacrylate). However, a prerequisite for efficient bonding of cyanoacrylates is a dry substrate. Such adhesives fail where there is pronounced bleeding.

As an alternative to the cyanoacrylates, biological adhesives such as, for example, BioGlue®, a mixture of glutaraldehyde and bovine serum albumin, various collagen- and gelatin-based systems (FloSeal®) and the fibrin adhesives (Tissucol) are available. Such systems are used primarily for stopping bleeding (haemostasis). In addition to the high costs, fibrin adhesives are distinguished by a relatively weak adhesive strength and rapid degradation, so that they can only be used in the case of relatively small injuries on unstretched tissue. Collagen- and gelatin-based systems such as FloSeal® are used solely for haemostasis. In addition, because fibrin and thrombin are obtained from human material and collagen and gelatin are obtained from animal material, there is always the risk of an infection in biological systems. Biological materials must additionally be stored in cool conditions, so that their use in emergency care, such as, for example, in disaster areas, in military campaigns, etc., is not possible. Traumatic wounds are treated in such cases with QuikClot® or QuikClot ACS+™, which is a mineral granulate that is introduced into the wound in an emergency and, by removing water, leads to coagulation. In the case of QuikClot®, this is a strongly exothermic reaction, which leads to burns. QuikClot ACS+™ is a gauze into which the salt is embedded. The system must be pressed firmly onto the wound in order to stop the bleeding.

The preparation and use of polyurea systems as tissue adhesives is known from WO 2009/106245 A2. The systems disclosed therein comprise at least two components. The components are an amino-functional aspartic acid ester and an isocyanate-functional prepolymer, which is obtainable by reaction of aliphatic polyisocyanates with polyester polyols. The described 2-component polyurea systems can be used as tissue adhesives for sealing wounds in human and animal cell structures. A very good adhesion result can thereby be achieved.

In order to ensure good miscibility of the two components of the polyurea system, the viscosity of the components at 23° C. should be less than 10,000 mPas where possible. Prepolymers having NCO functionalities of less than 3 exhibit such a correspondingly low viscosity. When such prepolymers are used, it is necessary to use as the second component an aspartic acid ester having an amino functionality of more than 2, because it is otherwise not possible to produce a polymer network. This is necessary, however, in order that the polyurea system, or a seam consisting thereof, has the desired mechanical properties such as elasticity and strength. Moreover, it is a disadvantage when using difunctional aspartic acid esters that the curing time is up to 24 hours, the polyurea system in many cases remaining tacky, that is to say is not tack-free, even after that time.

The object of the invention was, therefore, to provide an isocyanate-reactive component for a polyurea system, which isocyanate-reactive component is readily miscible with a prepolymer having an NCO functionality of less than 3, has an amino functionality of more than 2 and can be reacted quickly with the prepolymer to form a three-dimensional polyurea network. An additional condition to be taken into consideration was that the cured system does not have cytotoxicity according to ISO 10993 when used in humans or in animals.

The object is achieved according to the invention by a compound of formula (I)

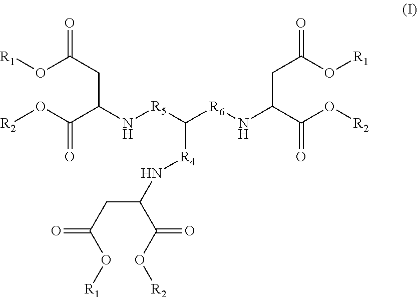

wherein
R₁, R₂ each independently of the other are identical or different organic radicals which do not contain Zerewitinoff-active hydrogen, and
R₄, R₅, R₆ each independently of the others are saturated, linear or branched organic radicals which do not contain Zerewitinoff-active hydrogen and which are also optionally substituted in the chain by heteroatoms.

The compound according to the invention can readily be mixed with a prepolymer because it has a viscosity of less than 10,000 mPas at 23° C. In addition, it has an amino functionality of 3 and is consequently able quickly to form a three-dimensional polyurea network with prepolymers having an NCO functionality of 2. The network is distinguished by high elasticity, strength, adhesive strength and an absence of cytotoxicity. Moreover, the network is no longer tacky, that is to say is tack-free, after only a short time.

In formula (I), the radicals R₄, R₅, R₆ each independently of the others can be linear or branched, in particular saturated, aliphatic C1 to C12, preferably C2 to C10, particularly preferably C3 to C8 and most particularly preferably C3 to C6 hydrocarbon radicals. Such amino-functional compounds are distinguished by the fact that they cure particularly quickly with prepolymers to form a highly adhesive, elastic and strong polyurea network.

According to a further preferred embodiment of the compound according to the invention, the radicals R₁, R₂ each independently of the other are linear or branched organic C1 to C10, preferably C1 to C8, particularly preferably C2 to C6, most particularly preferably C2 to C4 radicals and in particular are aliphatic hydrocarbon radicals. This compound too is distinguished by rapid network formation on reaction with a prepolymer.

It is likewise preferred for the radicals R₁ and R₂ each to be identical and/or the radicals R₄, R₅, R₆ each to be identical.

Most particular preference is given to a compound of formula (I) wherein R₁ and R₂ are each simultaneously methyl or ethyl and R₄, R₅, R₆ are each simultaneously ethyl, propyl or butyl.

The invention further provides a polyurea system comprising
as component A) isocyanate-functional prepolymers obtainable by reaction of
   aliphatic polyisocyanates A1) with
   polyols A2), which in particular can have a number-average molecular weight of ≧400 g/mol and a mean OH functionality of from 2 to 6,
as component B) an amino-functional compound of formula (I) according to the invention,
optionally as component C) organic fillers, which in particular can have a viscosity at 23° C., measured in accordance with DIN 53019, in the range of from 10 to 6000 mPas,
optionally as component D) reaction products of isocyanate-functional prepolymers according to component A) with amino-functional compounds according to component B) and/or organic fillers according to component C), and
optionally as component E) water and/or a tertiary amine.

The polyurea systems according to the invention are obtained by mixing the prepolymers A) with the amino-functional compound B) and optionally components C), D) and/or E). The ratio of free or blocked amino groups to free NCO groups is preferably 1:1.5, particularly preferably 1:1. Water and/or amine are thereby added to component B) or C).

The isocyanate-functional prepolymers A) are obtainable by reaction of polyisocyanates A1) with polyols A2), optionally with the addition of catalysts as well as auxiliary substances and additives.

As polyisocyanates A1) there can be used, for example, monomeric aliphatic or cycloaliphatic di- or tri-isocyanates such as 1,4-butylene diisocyanate (BDI), 1,6-hexamethylene diisocyanate (HDI), isophorone diisocyanate (IPDI), 2,2,4- and/or 2,4,4-trimethylhexamethylene diisocyanate, the isomeric bis-(4,4′-isocyanatocyclohexyl)-methanes or mixtures thereof of any desired isomer content, 1,4-cyclohexylene diisocyanate, 4-isocyanatomethyl-1,8-octane diisocyanate (nonane triisocyanate), and alkyl 2,6-diisocyanatohexanoate (lysine diisocyanate) having C1-C8-alkyl groups.

In addition to the monomeric polyisocyanates A1) mentioned above there can also be used the higher molecular weight secondary products thereof having a uretdione, isocyanurate, urethane, allophanate, biuret, iminooxadiazinedione or oxadiazinetrione structure and mixtures thereof.

Preference is given to the use of polyisocyanates A1) of the above-mentioned type having only aliphatically or cycloaliphatically bonded isocyanate groups or mixtures thereof.

It is likewise preferred for polyisocyanates A1) of the above-mentioned type having a mean NCO functionality of from 1.5 to 2.5, preferably of from 1.6 to 2.4, more preferably of from 1.7 to 2.3, most particularly preferably of from 1.8 to 2.2 and in particular of 2 to be used.

Hexamethylene diisocyanate is most particularly preferably used as the polyisocyanate A1).

According to a preferred embodiment of the polyurea system according to the invention, it is provided that the polyols A2) are polyester polyols and/or polyester-polyether polyols and/or polyether polyols. Particular preference is given to polyester-polyether polyols and/or polyether polyols having an ethylene oxide content of from 60 to 90 wt. %.

It is also preferred for the polyols A2) to have a number-average molecular weight of from 4000 to 8500 g/mol.

Suitable polyether ester polyols are prepared according to the prior art preferably by polycondensation from polycarboxylic acids, anhydrides of polycarboxylic acids and esters of polycarboxylic acids with readily volatile alcohols, preferably C1 to C6 monools, such as methanol, ethanol, propanol or butanol, with low molecular weight and/or higher molecular weight polyol in molar excess; wherein there are used as the polyol ether-group-containing polyols optionally in mixtures with other ether-group-free polyols.

Mixtures of the higher molecular weight and of the low molecular weight polyols can, of course, also be used for the polyether ester synthesis.

Such low molecular weight polyols in molar excess are polyols having molar masses of from 62 to 299 daltons, having from 2 to 12 carbon atoms and hydroxyl functionalities of at least 2, which can further be branched or unbranched and the hydroxyl groups of which are primary or secondary. These low molecular weight polyols can also contain ether groups. Typical representatives are ethylene glycol, 1,2-propanediol, 1,3-propanediol, 1,4-butanediol, 2,3-butanediol, 2-methyl-1,3-propanediol, 1,5-pentanediol, 1,6-hexanediol, 3-methyl-1,5-pentanediol, 1,8-octanediol, 1,10-decanediol, 1,12-dodecanediol, cyclohexanediol, diethylene glycol, triethylene glycol and higher homologues, dipropylene glycol, tripropylene glycol and higher homologues, glycerol, 1,1,1-trimethylolpropane, and oligo-tetrahydrofurans with hydroxyl end groups. Mixtures within this group can, of course, also be used.

Higher molecular weight polyols in molar excess are polyols having molar masses of from 300 to 3000 daltons, which can be obtained by ring-opening polymerisation of epoxides, preferably ethylene oxide and/or propylene oxide, as well as by acid-catalysed, ring-opening polymerisation of tetrahydrofuran. Either alkali hydroxides or double-metal-cyanide catalysts can be used for the ring-opening polymerisation of epoxides.

As starters for ring-opening epoxide polymerisations there can be used all at least bifunctional molecules from the group of the amines and the above-mentioned low molecular weight polyols. Typical representatives are 1,1,1-trimethylolpropane, glycerol, o-TDA, ethylenediamine, 1,2-propylene glycol, etc., as well as water, including mixtures thereof. Mixtures within the group of the excess higher molecular weight polyols can, of course, also be used.

The synthesis of the higher molecular weight polyols, in so far as hydroxyl-group-terminated polyalkylene oxides of ethylene oxide and/or propylene oxide are concerned, can be effected randomly or block-wise, it also being possible for mixed blocks to be present.

Polycarboxylic acids are both aliphatic and aromatic carboxylic acids, which can be both cyclic, linear, branched or unbranched and which can contain from 4 to 24 carbon atoms.

Examples are succinic acid, glutaric acid, adipic acid, azelaic acid, sebacic acid, 1,10-decanedicarboxylic acid, 1,12-dodecanedicarboxylic acid, phthalic acid, terephthalic acid, isophthalic acid, trimellitic acid, pyromellitic acid. Succinic acid, glutaric acid, adipic acid, sebacic acid, lactic acid, phthalic acid, terephthalic acid, isophthalic acid, trimellitic acid, pyromellitic acid are preferred. Succinic acid, glutaric acid and adipic acid are particularly preferred.

The group of the polycarboxylic acids also includes hydroxycarboxylic acids, or their inner anhydrides, such as, for example, caprolactone, lactic acid, hydroxybutyric acid, ricinoleic acid, etc. Also included are monocarboxylic acids, in particular those which have more than 10 carbon atoms, such as soybean oil fatty acid, palm oil fatty acid and groundnut oil fatty acid, wherein the proportion thereof in the whole of the reaction mixture constituting the polyether ester polyol is not more than 10 wt. % and, in addition, the accompanying low functionality is compensated for by the concomitant use of at least trifunctional polyols, whether it be on the side of the low molecular weight or high molecular weight polyols.

According to the prior art, the preparation of the polyether ester polyol is carried out at elevated temperature in the range from 120 to 250° C., initially under normal pressure, later with the application of a vacuum of from 1 to 100 mbar, preferably, but not necessarily, using an esterification or transesterification catalyst, the reaction being completed until the acid number falls to values of from 0.05 to 10 mg KOH/g, preferably from 0.1 to 3 mg KOH/g and particularly preferably from 0.15 to 2.5 mg KOH/g.

An inert gas can further be used during the normal pressure phase prior to the application of a vacuum. Of course, liquid or gaseous entrainers can also be used as an alternative or for individual phases of the esterification. For example, the water of reaction can be discharged equally as well when nitrogen is used as carrier gas as when an azeotropic entrainer, such as, for example, benzene, toluene, xylene, dioxane, etc., is used.

Mixtures of polyether polyols with polyester polyols in arbitrary ratios can, of course, also be used.

Polyether polyols are preferably polyalkylene oxide polyethers based on ethylene oxide and optionally propylene oxide.

Such polyether polyols are preferably based on difunctional or higher functional starter molecules such as difunctional or higher functional alcohols or amines.

Examples of such starters are water (regarded as a diol), ethylene glycol, propylene glycol, butylene glycol, glycerol, TMP, sorbitol, pentaerythritol, triethanolamine, ammonia or ethylenediamine.

Hydroxyl-group-containing polycarbonates, preferably polycarbonate diols, having number-average molecular weights Mₙ of from 400 to 8000 g/mol, preferably from 600 to 3000 g/mol, can likewise be used. They are obtainable by reaction of carbonic acid derivatives, such as diphenyl carbonate, dimethyl carbonate or phosgene, with polyols, preferably diols.

Examples of such diols are ethylene glycol, 1,2- and 1,3-propanediol, 1,3- and 1,4-butanediol, 1,6-hexanediol, 1,8-octanediol, neopentyl glycol, 1,4-bishydroxymethylcyclohexane, 2-methyl-1,3-propanediol, 2,2,4-trimethyl-1,3-pentanediol, dipropylene glycol, polypropylene glycols, dibutylene glycol, polybutylene glycols, bisphenol A and lactone-modified diols of the above-mentioned type.

For the preparation of the prepolymer A), the polyisocyanate A1) can be reacted with the polyol A2) with an NCO/OH ratio of preferably from 4:1 to 12:1, particularly preferably 8:1, and then the content of unreacted polyisocyanate can be separated off by suitable methods. Thin-film distillation is conventionally used for that purpose, prepolymers having residual monomer contents of less than 1 wt. %, preferably less than 0.1 wt. %, most particularly preferably less than 0.03 wt. %, being obtained.

During the preparation, stabilisers such as benzoyl chloride, isophthaloyl chloride, dibutyl phosphate, 3-chloropropionic acid or methyl tosylate can optionally be added.

The reaction temperature in the preparation of the prepolymers A) is preferably from 20 to 120° C. and more preferably from 60 to 100° C.

The prepolymers that are prepared have a mean NCO content, measured in accordance with DIN EN ISO 11909, of from 2 to 10 wt. %, preferably from 2.5 to 8 wt. %.

According to a further embodiment of the polyurea system according to the invention, the prepolymers A) can have a mean NCO functionality of from 1.5 to 2.5, preferably of from 1.6 to 2.4, more preferably of from 1.7 to 2.3, most particularly preferably of from 1.8 to 2.2 and in particular of 2.

The organic fillers of component C) can preferably be hydroxy-functional compounds, in particular polyether polyols having repeating ethylene oxide units.

It is also advantageous for the fillers of component C) to have a mean OH functionality of from 1.5 to 3, preferably of from 1.8 to 2.2 and particularly preferably of 2.

For example, there can be used as organic fillers polyethylene glycols that are liquid at 23° C., such as PEG 200 to PEG 600, their mono- or di-alkyl ethers such as PEG 500 dimethyl ether, liquid polyether and polyester polyols, liquid polyesters such as, for example, Ultramoll (Lanxess AG, Leverkusen, DE), and glycerol and its liquid derivatives such as, for example, triacetin (Lanxess AG, Leverkusen, DE).

The viscosity of the organic fillers, measured in accordance with DIN 53019 at 23° C., is preferably from 50 to 4000 mPas, particularly preferably from 50 to 2000 mPas.

In a preferred embodiment of the polyurea system according to the invention, polyethylene glycols are used as organic fillers. They preferably have a number-average molecular weight of from 100 to 1000 g/mol, particularly preferably from 200 to 400 g/mol.

In order further to reduce the mean equivalent weight of the compounds used overall for the prepolymer crosslinking, based on the NCO-reactive groups, it is additionally possible to prepare reaction products of the prepolymers A) with the amino-functional compound B) and/or the organic fillers C), provided they are amino- or hydroxy-functional, in a separate preliminary reaction and then use them as the higher molecular weight curing agent component.

Preferably, ratios of isocyanate-reactive groups to isocyanate groups of from 50 to 1 to 1.5 to 1, particularly preferably from 15 to 1 to 4 to 1, are established in the pre-extension.

The advantage of this modification by pre-extension is that the equivalent weight and the equivalent volume of the curing agent component can be modified within wider limits. Commercially available 2-chamber metering systems can accordingly be used for the application, in order to obtain an adhesive system which, with existing chamber volume ratios, can be adjusted to the desired ratio of NCO-reactive groups to NCO groups.

According to a further embodiment of the polyurea system according to the invention, it is provided that component E) comprises a tertiary amine of the general formula (II)

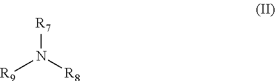

wherein

R₇, R₈, R₉ independently of one another can be alkyl or heteroalkyl radicals having heteroatoms in the alkyl chain or at the end thereof, or R₇ and R₈ together with the nitrogen atom carrying them can form an aliphatic, unsaturated or aromatic heterocycle which can optionally contain further heteroatoms.

These polyurea systems are distinguished by particularly rapid curing.

The compounds used in component E) can most particularly preferably be tertiary amine selected from the group triethanolamine, tetrakis(2-hydroxyethyl)ethylenediamine, N,N-dimethyl-2-(4-methylpiperazin-1-yl)ethanamine, 2-{[2-(dimethylamino)ethyl](methyl)amino}-ethanol, 3,3′,3″-(1,3,5-triazinane-1,3,5-triyl)tris(N,N-dimethyl-propan-1-amine).

Very particularly high curing speeds can also be achieved if component E) comprises from 0.2 to 2.0 wt. % water and/or from 0.1 to 1.0 wt. % of the tertiary amine.

It is, of course, also possible to incorporate into the polyurea systems pharmacologically active ingredients such as analgesics with and without anti-inflammatory activity, antiphlogistics, substances having antimicrobial activity, antimycotics, substances having antiparasitic activity.

The polyurea system according to the invention is suitable in particular for sealing, bonding, gluing or covering cell tissue and in particular for stopping the escape of blood or tissue fluids or for sealing leaks in cell tissue. Most particularly preferably, it can be used for the production of an agent for sealing, bonding, gluing or covering human or animal cell tissue. By means of the polyurea system according to the invention it is possible to produce rapidly curing, transparent, flexible and biocompatible seams which adhere firmly to the tissue.

The invention further provides a metering system having two chambers for a polyurea system according to the invention, in which one chamber contains component A) and the other chamber contains component B) and optionally components C), D) and E) of the polyurea system. Such a metering system is suitable in particular for applying the polyurea system as an adhesive to tissue.

### EXAMPLES

The invention is explained in greater detail below by means of examples.

### Methods

Molecular weight: The molecular weights were determined by means of gel permeation chromatography (GPC) as follows: Calibration is carried out using polystyrene standards with molecular weights of Mp 1,000,000 to 162. Tetrahydrofuran p.A. was used as eluant. The following parameters were observed during the double measurement: degassing: online degasser; flow rate: 1 ml/min; analysis time: 45 minutes; detectors: refractometer and UV detector; injection volume: 100 μl-200 μl. Calculation of the molar mass means M_{w}; Mₙ and Mₚ and of the polydispersity M_{w}/Mₙ was carried out with software assistance. Baseline points and evaluation limits were fixed in accordance with DIN 55672 Part 1.

NCO content: Unless expressly mentioned otherwise, the NCO content was determined volumetrically in accordance with DIN-EN ISO 11909.

Viscosity: The viscosity was determined in accordance with ISO 3219 at 23° C.

Residual monomer content: The residual monomer content was determined in accordance with DIN ISO 17025.

NMR: The NMR spectra were recorded using a Bruker DRX 700 device.

### Substances

Polyethylene glycol 600 (Aldrich)

### Synthesis of tetraethyl 2,2′-(4-(3-(1,4-diethoxy-1,4-dioxobutan-2-ylamino)propyl)heptane-1,7-diyl)bis(azandiyl)disuccinate (3)

### 4-(2-Cyanoethyl)heptanedinitrile (1)

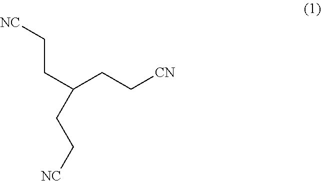 [SMILES: N#CCCC(CCC#N)CCC#N]

A mixture of 12.2 g (55.4 mmol) of 4-(2-cyanoethyl)-4-nitroheptanedinitrile and 18 ml of tributyltin dihydride was heated at reflux overnight with 2.78 g of azo-bis-(isobutyronitrile) (AIBN) in 250 ml of acetonitrile. After removal of the solvent in vacuo, the product was stirred with n-hexane and crystallised with a mixture of n-hexane and dichloromethane. 8.25 g (85%) of the product were obtained in the form of a colourless solid.

### 4-(3-Aminopropyl)heptane-1,7-diamine (2)

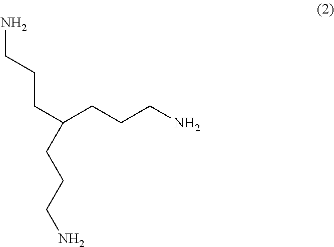 [SMILES: NCCCC(CCCN)CCCN]

8 g (45 7 mmol) of 4-(2-cyanoethyl)heptanedinitrile were hydrogenated for 5 hours at 130° C. and a hydrogen pressure of 100 bar in 100 ml of 7N methanolic ammonia solution. After cooling to room temperature, the reaction mixture was filtered over kieselguhr and washed with methanol. After removal of the solvent in vacuo, 7.5 g of the crude product were obtained and were used in the next stage without being purified further.

### Tetraethyl 2,2′-(4-(3-(1,4-diethoxy-1,4-dioxobutan-2-ylamino)propyl)heptane-1,7-diyl)bis-(azandiyl)disuccinate (3)

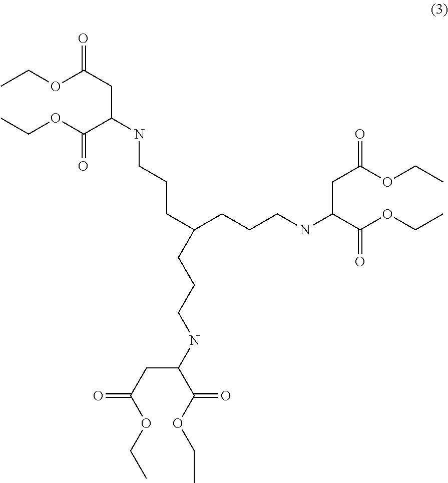 [SMILES: CCOC(=O)CC(NCCCC(CCCNC(CC(=O)OCC)C(=O)OCC)CCCNC(CC(=O)OCC)C(=O)OCC)C(=O)OCC]

25.7 g (150 mmol) of maleic acid diethyl ester were added to a solution of 7.5 g (40 mmol) of 4-(3-aminopropyl)heptane-1,7-diamine in 100 ml of ethanol. The reaction mixture was stirred for 3 days at 60° C. After removal of the solvent in vacuo, the product was purified by column chromatography (hexane/ethyl acetate 1:1, then dichloromethane/methanol 10:1). 15.5 g (39%) of the product were obtained in the form of a yellow oil.

¹H-NMR (CDCl₃, 700 MHz): δ=1.27 (t, 9H), 1.28 (m, 6H), 1.29 (t, 9H), 1.42 (m, 6H) 1.72 (s, 3NH), 2.48 (m, 1H), 2.61, (m, 6H), 2.64 (dd, 6H), 3.60 (t, 3H), 4.19 (q, 6H), 4.2 (q, 6H).

¹³C-NMR (CDCl₃, 700 MHz): 13.93, 13.99, 26.9, 30.6, 36.9, 37.9, 48.3, 57.6, 60.4, 60.9, 170.6, 173.4.

### Synthesis of Prepolymer A

212.5 g (1.8 mol) of succinic acid were heated to 235° C., with stirring, with 1591.5 g of polyethylene glycol 600 (2.6 mol). The water that formed was distilled off for a period of 8.5 hours. 100 ppm of tin(II) chloride were then added, and heating at 235° C. was continued for a further 9 hours in vacuo (15 mbar) in a water separator.

672 g of HDI (4 mol) were placed in a reaction vessel with 0.1 wt. % benzoyl chloride and heated to 80° C. 788 g of the polyester were then metered in, with stirring, over a period of one hour, and stirring was continued at 80° C. until a constant NCO content was reached. Excess HDI was removed at 140° C. and 0.13 mbar by means of a thin-film evaporator. The resulting prepolymer had an NCO content of 3.5% and a viscosity of 4700 mPas/23° C. The residual monomer content was <0.03% HDI.

### Synthesis of Prepolymer B

A prepolymer was prepared analogously to prepolymer A from 263 g (1.8 mol) of adipic acid and 1591.5 g of polyethylene glycol 600 (2.6 mol). The resulting prepolymer had an NCO content of 5.93% and a viscosity of 1450 mPas/23° C. The residual monomer content was <0.03% HDI.

### Synthesis of Prepolymer C

465 g of HDI and 2.35 g of benzoyl chloride were placed in a one-litre four-necked flask. Within a period of 2 hours, 931.8 g of a polyether having an ethylene oxide content of 71% and a propylene oxide content of 29% (in each case based on the total alkylene oxide content) were added at 80° C., and stirring was then carried out for one hour. Excess HDI was then distilled off by thin-film distillation at 130° C. and 0.13 mbar. 980 g (71%) of a prepolymer having an NCO content of 2.37% and a viscosity of 4500 mPas/23° C. were obtained. The residual monomer content was <0.03% HDI.

### Production of the Tissue Adhesive

4 g of the prepolymer in question were stirred thoroughly in a beaker with an equivalent amount of the amino-functional compound 3 which had been prepared Immediately thereafter, the polyurea system was applied as a thin layer to the muscle tissue to be bonded. The time for which the polyurea system had a sufficiently low viscosity that it could be applied to the tissue without difficulty was determined as the processing time.

The time after which the polyurea system was no longer tacky (tack free time) was measured by adhesion tests using a glass rod. To that end, the glass rod was brought into contact with the layer of the polyurea system. When the rod no longer adhered, the system was considered to be tack-free. In addition, the adhesive force was determined by coating two pieces of muscle tissue (1=4 cm, h=0.3 cm, b=1 cm) with the polyurea system 1 cm from the ends and adhesively bonding them so that they overlapped. The adhesive force of the polyurea system was tested in each case by pulling.

| | Curing | | | | | Adhesive |
|---|---|---|---|---|---|---|
| | agent | Processing time | | Tack free time | | force |
| Prepolymer A | 3 | 1 | min 30 s | 2 | min | ++ |
| Prepolymer B | 3 | 1 | min | 2 | min | + |
| Prepolymer B | 4 | 8 | min | >30 | min | + |
| (Comparison | | | | | | |
| Example 1) | | | | | | |
| Prepolymer A | 5 | 3 | min | 5 | min | ++ |

++: very good;
+: good

### Comparison Example 1: Difunctional Prepolymer+Difunctional Curing Agent

Instead of the amino-functional compound 3 which had been prepared, tetraethyl 2,2′-[(2-methylpentane-1,5-diyl)diimino]dibutanoate (4) described in EP 2 145 634 was reacted with prepolymer B. The processing time here was 8 minutes. The polyurea system was still not tack-free even after 10 minutes.

### Measurement of the Cytotoxicity of an Adhesive Produced with (3)

Prepolymer C was reacted with an equivalent amount of 3. The cytotoxicity was measured in accordance with ISO 10993-5:2009 using L929 cells. There was no reduction in cell viability. Accordingly, the polyurea system is not to be categorised as cytotoxic.

### Comparison Example 2: Cytotoxicity of a Structurally Similar Trifunctional Curing Agent

Diethyl 2-[(8-[(1,4-diethoxy-1,4-dioxobutan-2-yl)amino]-4-{[(1,4-diethoxy-1,4-dioxobutan-2-yl)-amino]methyl}octyl)amino]butanedioate (5)

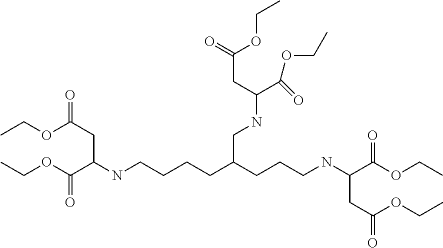 [SMILES: CCOC(=O)CC(NCCCCC(CCCNC(CC(=O)OCC)C(=O)OCC)CNC(CC(=O)OCC)C(=O)OCC)C(=O)OCC]

346 g (2 mol) of maleic acid diethyl ester were added dropwise to 115.3 g (0.6 mol) of triaminononane. The reaction mixture was heated at 60° C. for 3 days until no further maleic acid ester was detectable (Bayer reagent).

### Measurement of the Cytotoxicity of the Cured Adhesive

4 g of prepolymer C were stirred thoroughly in a beaker with an equivalent amount of (5) and cured. The adhesive was measured in accordance with ISO 10993-5:2009 using L929 cells. The cell viability fell to 4% (high cyctotoxicity).

1-16. (canceled)

17. A compound of formula (I) 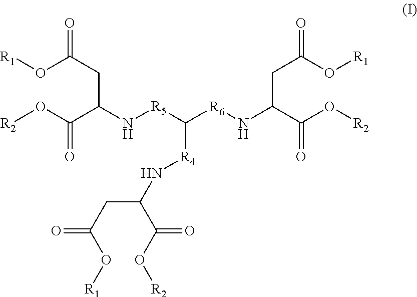
wherein
R₁ and R₂ each independently of the other are identical or different organic radicals which do not contain Zerewitinoff-active hydrogen, and
R₄, R₅ and R₆ each independently of the others are saturated, linear or branched organic radicals which do not contain Zerewitinoff-active hydrogen and which are also optionally substituted in the chain by heteroatoms.

18. The compound according to claim 17, wherein the radicals R₄, R₅ and R₆ each independently of the others are linear or branched, saturated, aliphatic C1 to C12 hydrocarbon radicals.

19. The compound according to claim 17, wherein the radicals R₄, R₅ and R₆ each independently of the others are saturated, aliphatic C3 to C6 hydrocarbon radicals.

20. The compound according to claim 17, wherein the radicals R₁ and R₂ each independently of the other are linear or branched organic C1 to C10.

21. The compound according to claim 17, wherein the radicals R₁ and R₂ each independently of the other are linear or branched C2 to C4 aliphatic hydrocarbon radicals.

22. The compound according to claim 17, wherein the radicals R₁ and R₂ are each identical and/or the radicals R₄, R₅, R₆ are each identical.

23. A polyurea system comprising
as component A) isocyanate-functional prepolymers obtainable by reaction of
aliphatic polyisocyanates A1) with
polyols A2),
as component B) the amino-functional compound according to claim 17,
optionally as component C) organic fillers, which in particular can have a viscosity at 23° C., measured in accordance with DIN 53019, in the range of from 10 to 6000 mPas,
optionally as component D) reaction products of isocyanate-functional prepolymers according to component A) with amino-functional compounds according to component B) and/or organic fillers according to component C), and
optionally as component E) water and/or a tertiary amine.

24. The polyurea system as claimed in claim 23, wherein said polyols A2) have a number-average molecular weight of ≧400 g/mol and an average OH functionality of 2 to 6.

25. The polyurea system according to claim 23, wherein the polyols A2) comprise polyester polyols and/or polyester-polyether polyols and/or polyether polyols and/or polyether polyols having an ethylene oxide content of from 60 to 90 wt. %.

26. The polyurea system according to claim 23, wherein the polyols A2) comprise polyester-polyether polyols and/or polyether polyols having an ethylene oxide content of from 60 to 90 wt. %.

27. The polyurea system according to claim 23, wherein the polyols A2) have a number-average molecular weight of from 4000 to 8500 g/mol.

28. The polyurea system according to claim 23, wherein the prepolymers A) have a mean NCO functionality of from 1.5 to 2.5.

29. The polyurea system according to claim 23, wherein the prepolymers A) have a mean NCO functionality of 2.

30. The polyurea system according to claim 23, wherein the organic fillers of component C) are hydroxy-functional compounds having repeating ethylene oxide units.

31. The polyurea system according to claim 25, wherein the fillers of component C) have a mean OH functionality of from 1.5 to 3.

32. The polyurea system according to claim 23 wherein component E) comprises a tertiary amine of the general formula (II) 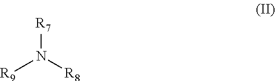
wherein
R₇, R₈, R₉ independently of one another can be alkyl or heteroalkyl radicals having heteroatoms in the alkyl chain or at the end thereof, or R₇ and R₈ together with the nitrogen atom carrying them can form an aliphatic, unsaturated or aromatic heterocycle which can optionally contain further heteroatoms.

33. The polyurea system according to claim 23, wherein the tertiary amine is selected from the group consisting of triethanolamine, tetrakis(2-hydroxyethyl)ethylenediamine, N,N-dimethyl-2-(4-methylpiperazin-1-yl)ethanamine, 1-{[2-(dimethylamino)ethyl](methyl)amino}ethanol and 3,3′,3″-(1,3,5-triazinane-1,3,5-triyl)tris(N,N-dimethyl-propan-1-amine).

34. The polyurea system according to claim 23, wherein component E) comprises from 0.2 to 2.0 wt. % water and/or from 0.1 to 1.0 wt. % of the tertiary amine.

35. The polyurea system according to claim 23 for sealing, bonding, gluing or covering cell tissue, for stopping the escape of blood or tissue fluids or for sealing leaks in cell tissue.

36. The polyurea system according to claim 30 for use for sealing, bonding, gluing or covering human or animal cell tissue.

37. A metering system comprising two chambers for the polyurea system according to claim 21, wherein one chamber contains component A) and the other chamber contains component B) and optionally components C), D) and E) of the polyurea system.
